(19) United States
(12) Patent Application Publication
(10) Pub. No.: US 2025/0179297 A1
(43) Pub. Date: Jun. 5, 2025

(54) INTRINSICALLY CONDUCTING ELASTOMERS AND METHODS OF MAKING THE SAME

(71) Applicant: Meta Platforms Technologies, LLC, Menlo Park, CA (US)

(72) Inventors: Li Yao, Bothell, WA (US); Dan My Nguyen, Newark, DE (US)

(21) Appl. No.: 18/960,445

(22) Filed: Nov. 26, 2024

Related U.S. Application Data
(60) Provisional application No. 63/605,009, filed on Dec. 1, 2023.

(51) Int. Cl. C08L 81/08; A61B 5/268; A61B 5/296; C08J 5/18; C08L 53/00

(52) U.S. Cl. CPC C08L 81/08; A61B 5/268; A61B 5/296; C08J 5/18; C08L 53/005; C08J 2353/00; C08J 2381/08; C08J 2453/00; C08J 2481/08; C08L 2203/02; C08L 2205/03

(57) ABSTRACT

Described herein are compositions for tunable intrinsically conducting elastomers and methods of making and using the same. The compositions include a conductive polymer, a polymer counterion, and optionally a bottlebrush block copolymer. Optionally, the compositions include a crosslinker and a photoinitiator. Also described herein are copolymer blends, elastomeric materials, and films including any one of the intrinsically conducting elastomers.

### CROSS-REFERENCES TO RELATED APPLICATION

This application claims priority to U.S. Provisional Application No. 63/605,009, filed on Dec. 1, 2023, which is incorporated herein by reference in its entirety.

### BACKGROUND

Bioelectronic devices, including implantable and wearable devices, are interfaced with the human body to measure physiological signals or to provide electrical stimulation for treatment. While human body/skin is soft and stretchable, conventional electronics are usually hard and rigid. The mismatch between these two significantly hampers the performance and efficacy of bioelectronics' stimulation and recording. For example, metal electrodes are very rigid with high bending modulus, and are not comfortable to be worn all day long.

### SUMMARY

Described herein are intrinsically conductive elastomers and methods of making the materials. Generally, the intrinsically conductive elastomers are based on doped conductive polymers with additives to enhance the electrical conductivity, softness and stretchability, which may be suitable for bioelectronic applications such as electrodes for biopotential signal sensing. Unlike traditional composite materials, conductors made from intrinsically conducting elastomers described herein have potential applications in biopotential signal sensing, such as electrocardiogram (ECG), electroencephalogram (EEG), and electromyography (EMG) electrodes. Specifically, the designed blends of these components as described herein can be used to provide materials with good mechanical properties, high conductivity, and other properties to enable biopotential signal acquisition, such as low skin contact impedance.

Described herein are intrinsically conductive elastomer compositions. The intrinsically conductive elastomer compositions can include of one or more conductive polymers, such as poly(3,4-ethylenedioxythiophene) (PEDOT), polypyrrole (PPy), and/or polyaniline (PANI), and one or more polymer counterions, such as, but not limited to, polystyrene sulfonate (PSS). Optionally the compositions may include and additional additives as dopants or tougheners, such as polyethylene glycol, ethylene glycol, and others. Optionally, the compositions can further comprise one or more polymer additives, such as, but not limited to, bottlebrush block copolymers. Optionally, the compositions can further comprise one or more crosslinkers and/or one or more photoinitiators. The intrinsically conductive elastomers may be cured in molds to form free-standing thin films, or coated or deposited onto conductive substrates, exhibiting desirable mechanical properties (e.g., softness, stretchability), electrical properties (e.g., mixed ionic-electronic conductivities), and low skin contact impedance for applications such as EMG electrodes.

Also described herein are copolymer blends that may include one or more conductive polymers and one or more polymer counterions. Optionally, the copolymer blends may further include a bottlebrush block copolymer.

Films of the copolymer blends described herein are also provided. In some examples, the films can be thin films. In some examples, the films may exhibit an electrical conductivity of 0.1 S cm⁻¹ to 1000 S cm⁻¹ S cm⁻¹. In some examples, the films may exhibit an electrical conductivity of 150 S cm⁻¹. In some examples, the films may exhibit skin-contact impedance of 0.2 Ohm to 2 MOhm. In some examples, the films may exhibit a skin-contact impedance of 0.40 MOhm. In some examples, the films may exhibit a strain at break of 2% to 50%. In some examples, the films exhibit up to 20% strain at break.

Provided also herein are elastomeric materials including any one of the compositions or copolymer blends described herein. Objects, coatings, and wearable devices including one or more of the compositions or copolymer blends are also provided.

Elastomeric materials described herein can be used as electrodes for biopotential sensors, and in some examples, for EMG sensors.

The details of one or more embodiments are set forth in the drawings and the description below. Other features, objects, and advantages will be apparent from the description and drawings, and from the claims.

### BRIEF DESCRIPTION OF THE DRAWINGS

FIG. 1 provides a schematic of one nonlimiting example of a copolymer blend described herein.

FIG. 2 provides a nonlimiting example of a crosslinker described herein.

FIG. 3 provides a plot showing the tensile strength of some nonlimiting examples of films as described herein.

FIG. 4 provides a graph showing the conductivity of some nonlimiting examples of films as described herein.

FIG. 5 provides a schematic of a nonlimiting example of a copolymer blend described herein.

FIG. 6 provides a plot showing the tensile strength of some nonlimiting examples of films as described herein.

### DETAILED DESCRIPTION

### 1. Compositions for Intrinsically Conductive Elastomers

Described herein are intrinsically conductive elastomer compositions including one or more conductive polymers and one or more polymer counterions, wherein the weight ratio of the conductive polymer and polymer counterion is from 1:10 to 10:1. In some examples, the polymer counterion is a block copolymer counterion.

In some examples, the weight ratio the conductive polymer to the polymer counterion in the composition is 1:10 to 10:1 (e.g., 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, or 10:1). In some examples, the weight ratio of the conductive polymer to the polymer counterion is 1:5 to 5:1 (e.g., 1:5, 1:2.5, 1:1, 2.5:1, or 5:1).

Conductive polymers, or intrinsically conductive polymers, refers to conjugated polymers doped by oxidation to remove some of the delocalized electrons. Conductive polymers may include conjugated polymer chains, such as, aromatic cycles. Optionally, the conductive polymers may include polymer chains such as polynaphthalenes, polypyrenes, and/or polyphenylenes. In some examples, the conductive polymers may include polymer chains containing conjugated double bonds, such as poly(acetylene)s, and/or alternating aromatic cycles and double bonds, such as poly(p-phenylene vinylene) (PPV). In some examples, the conductive polymers may include aromatic cycles containing nitrogen (N), such as poly(pyrroles) (PPY), polycarbazoles, polyindoles, polyazepines, and/or PANI. In some examples, the conductive polymers may include aromatic cycles containing sulfur(S), such as poly(thiophene)s (PT), poly(3,4-ethylenedioxythiophene), poly(p-phenylene sulfide) (PPS), etc. In some examples, the conductive polymers further include one or more side chains including additional functional groups. In some examples, the additional functional groups on the side chains of the conductive polymers include thiols, carboxylic acids, amines, hydroxyls, ionic groups, or a combination thereof. In some examples, the conductive polymers may include ionic functional groups attached to the side chains of the conjugated backbone of the polymer chains, such as sodium sulfonate, and others. In some examples, the conductive polymer includes poly(3,4-ethylenedioxythiophene) (PEDOT), polypyrrole (PPy), polyaniline (PANI), or a combination thereof. In some examples, the conductive polymer is poly(3,4-ethylenedioxythiophene) (PEDOT). In some examples, a weight-average molecular weight (M_{w}) of the conductive polymer is from 2,000 to 500,000 Da (e.g., from 2,000 to 250,000, from 10,000 to 125,000, or from 20,000 to 50,000). In some examples, the weight-average molecular weight (M_{w}) of the conductive polymer is from 2,000 to 200,000 Da (e.g., from 2,000 to 100,000, from 5,000 to 50,000, or from 10,000 to 25,000).

The polymer counterions described herein include polymers having the opposite charge as the conductive polymers included in the composition. For example, when the conductive polymer is PEDOT, a suitable polymer counterion may include, but is not limited to, PSS. In some examples, the polymer counterion can be grafted with additional blocks, and may include, for example, PEG and/or PEGMA. For example, a suitable block copolymer counterion may include, but is not limited to, PEG-b-PSS. In some examples, the block copolymer counterion can have functional end chain which can be further crosslinked, such as a thiol (SH) end chain. In some examples, the block copolymer counterion is P(SS-b-PEG) and includes a thiol chain end P(SS-b-PEG)-SH. In some examples, the block copolymer counterion may be prepared by a living polymerization, such as reversible addition-fragmentation chain transfer (RAFT) polymerization. Subsequently, and without being limited by theory, the SH-PSS-b-PEG can act as a matrix for the oxidative polymerization of 3,4-ethylenedioxythiophene (EDOT), to prepare an intrinsically conducting elastomer, such as PEDOT:P(SS-b-PEG)-SH. In some examples, the block copolymer counterion includes one or more of poly(styrene sulfonate) (PSS), a polyethylene glycol-based polymer (PEG) (e.g., poly(ethylene glycol) 4-cyano-4-(phenylcarbonothioylthio)pentanoate), poly(ethylene oxide)monomethacrylate (PEGMA), (P(SS-b-PEG)), or a combination thereof. In some examples, the block copolymer counterion includes PSS and PEG. In some examples, the block copolymer counterion includes PSS, PEG, and PEGMA. In some examples, the block copolymer counterion is (P(SS-b-PEG)).

In some examples, a weight-average molecular weight (M_{w}) of the polymer counterion or block copolymer counterion is between 2,000 and 500,000 Da. In some examples, a weight-average molecular weight (M_{w}) of the polymer counterion or block copolymer counterion is between 2,000 and 200,000 Da. In some examples, the polymer counterion or block copolymer counterion is from about 0.5 weight percent to about 25 weight percent (wt. %) of the composition, (e.g., 1.0 wt. %, 5.0 wt. %, 10 wt. %, 15 wt. %, or 20 wt. %). In some examples, the polymer counterion or block copolymer counterion is from about 0.1 wt. % to about 5.0 wt. % of the composition, (e.g., 0.1 wt. %, 0.5 wt. %, 1.0 wt. %, 1.5 wt. %, 2.0 wt. %, 2.5 wt. %, 3.0 wt. %, 3.5 wt. %, 4.0 wt. %, or 4.5 wt. %).

In some examples, the composition may further include a bottlebrush block copolymer. A bottlebrush block copolymer refers to a high-density side-chain-grafted block copolymer with high molecular weights (MWs), in which one or more polymeric side chains are tethered to each repeating unit of a linear polymer backbone, such that these block copolymers look like “bottlebrushes.” See Li et al., Bottlebrush polymers: From controlled synthesis, self-assembly, properties to applications, Progress in Polymer Science, Vol. 116, 101387 (2021), which is incorporated by reference herein in its entirety. In some examples, the bottlebrush block copolymer includes P(PEGMA-b-PEG) (poly(poly(ethylene glycol)methacrylate-b-poly(ethylene glycol)), which can be used as a secondary dopant to induce the aggregation of PEDOT to enhance conductivity. In some examples, the bottlebrush block copolymer can have functional end chain which can be further crosslinked (e.g., a crosslinkable chain end), such as a thiol (SH) end chain. In some examples, the bottlebrush block copolymer containing a thiol chain end is (P(PEGMA-b-PEG)-SH). Without being limited by theory, the PEGMA block of the bottlebrush block copolymer can provide softness, stretchability to a composition described herein, as well as compatibility with the human body. In some examples, the P(PEGMA-b-PEG) includes one or more thiol chain ends (P(PEGMA-b-PEG)-SH). In some examples, (P(PEGMA-b-PEG)-SH) is prepared by treating P(PEGMA-b-PEG) with a reducing agent (e.g., NaBH₄), and an alkylphosphine (e.g., tributylphosphine (PBu₃)). In some examples, the (P(PEGMA-b-PEG)-SH) has the structure of Compound 1 below, wherein m, n, and p are independently selected from 1 to 5,000 (e.g., from 1 to 2,500, from 100 to 1,250, or from 500 to 1,000).

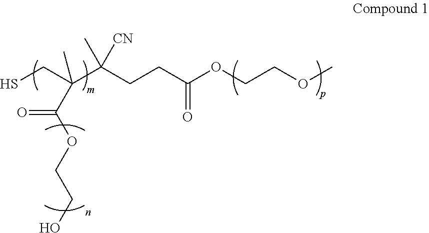

In some examples, a weight-average molecular weight (M_{w}) of the bottlebrush block copolymer is from 5,000 to 500,000 Da (e.g., from 5,000 to 250,000, from 10,000 to 125,000, or from 20,000 to 50,000).

The compositions described herein may further include a one or more crosslinkers. In some examples, the one or more crosslinkers includes one or more PEG vinyl ether crosslinkers. In some examples, the PEG vinyl ether crosslinker is poly(ethylene glycol)tetravinyl ether, as shown in FIG. 2. In some examples, the weight percent of the crosslinker in the composition is from about 0.05 wt. % to about 10 wt. % (e.g., 0.1 wt. %, 0.5 wt. %, 1.0 wt. %, 1.5 wt. %, 2.0 wt. %, 2.5 wt. %, 3.0 wt. %, 3.5 wt. %, 4.0 wt. %, 4.5 wt. %, 5.0 wt. %, 5.5 wt. %, 6.0 wt. %, 6.5 wt. %, 7.0 wt. %, 7.5 wt. %, 8.0 wt. %, 8.5 wt. %, 9.0 wt. %, or 9.5 wt. %).

The compositions described herein may also include one or more photoinitiators. The one or more photoinitiators may include a free radical generating photoinitiator. In some examples, the free radical photoinitiator is 2-hydroxy-4′-(2-hydroxyethoxy)-2-methylpropiophenone. In some examples, the weight percent of the photoinitiator in the composition is from about 0.0025 wt. % to about 10 wt. % (e.g., 0.005 wt. %, 0.01 wt. %, 0.05 wt. %, 0.1 wt. %, 0.5 wt. %, 1.0 wt. %, 1.5 wt. %, 2.0 wt. %, 2.5 wt. %, 3.0 wt. %, 3.5 wt. %, 4.0 wt. %, 4.5 wt. %, 5.0 wt. %, 5.5 wt. %, 6.0 wt. %, 6.5 wt. %, 7.0 wt. %, 7.5 wt. %, 8.0 wt. %, 8.5 wt. %, 9.0 wt. %, or 9.5 wt. %).

In some examples, the molar ratio of the PEG vinyl ether cross linker to the photoinitiator is from 1:1 to 5:1 (e.g., 1:1, 1.5:1, 2:1, 2.5:1, 3:1, 3.5:1, 4:1, 4.5:1, or 5:1).

Provided also herein is a copolymer blend including a conductive polymer including one or more of poly(3,4-ethylenedioxythiophene) (PEDOT), polypyrrole (PPy), polyaniline (PANI), or a combination thereof; and a copolymer counterion including one or more of poly(styrene sulfonate) (PSS), a polyethylene glycol-based polymer (PEG) (e.g., poly(ethylene glycol) 4-cyano-4 (phenylcarbonothioylthio)pentanoate), poly(ethylene oxide)monomethacrylate (PEGMA), (P(SS-b-PEG)), or a combination thereof. Optionally, the polymer counterion is a block copolymer counterion. In some examples, the conductive polymer in the copolymer blend includes PEDOT.

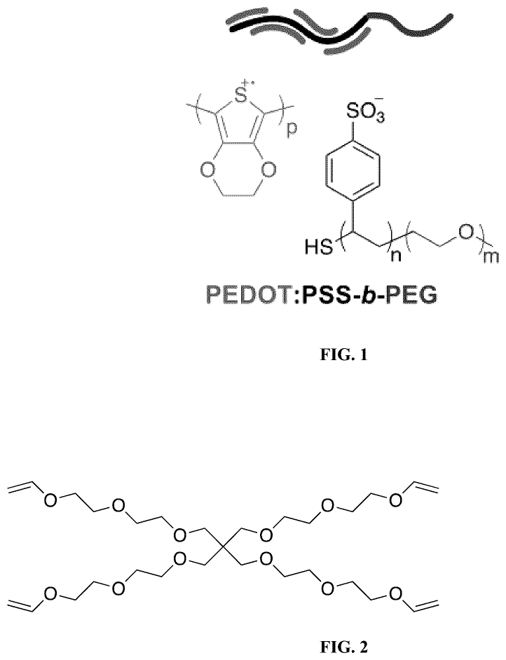

In some examples, the copolymer blend includes PEDOT:P(SS-b-PEG), as shown in one example in FIG. 1. In some examples, the copolymer blend includes PEDOT:P(SS-b-PEG)-SH, and is crosslinked with a PEG vinyl ether. In some examples, the PEG vinyl ether is poly(ethylene glycol)tetravinyl ether.

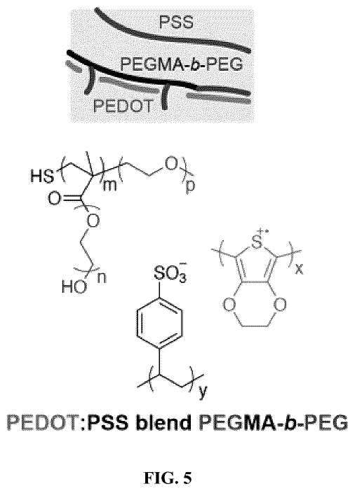

In some examples, the copolymer blend further includes a bottlebrush block copolymer. In some examples, the bottlebrush block copolymer includes PEGMA-b-PEG. In some examples, the bottlebrush block copolymer is P(PEGMA-b-PEG)-SH. In some examples, the bottlebrush block copolymer can be blended with conductive polymers and polymer counterions, such as PEDOT:PSS, at various ratios, as shown in FIG. 5.

In some examples, the conductive polymer, polymer counterion, and/or bottlebrush block copolymer includes a reactive chain end, such as a thiol chain end. In some examples, the thiol chain end is crosslinked with a PEG vinyl ether, which optionally may include poly(ethylene glycol)tetravinyl ether, as shown in FIG. 2.

In some examples, a weighted average molecular weight (M_{w}) of the conductive polymer of the copolymer blend is from 2,000 and 500,000 Da (e.g., from 2,000 to 250,000, from 10,000 to 125,000, or from 20,000 to 50,000). In some examples, a weighted average molecular weight (M_{w}) of the conductive polymer of the copolymer blend is from 2,000 to 200,000 Da (e.g., from 4,000 to 100,000 Da, from 8,000 to 50,000 Da, or from 16,000 to 25,000 Da).

In some examples, a weighted average molecular weight (M_{w}) of the block copolymer counterion of the copolymer blend is from 2,000 and 500,000 Da (e.g., from 2,000 to 250,000, from 10,000 to 125,000, or from 20,000 to 50,000). In some examples, a weighted average molecular weight (M_{w}) of the block copolymer counterion of the copolymer blend is from 2,000 to 200,000 Da (e.g., from 4,000 to 100,000 Da, from 8,000 to 50,000 Da, or from 16,000 to 25,000 Da).

In some examples, a weighted average molecular weight (M_{w}) of the bottlebrush block copolymer of the copolymer blend is from 5,000 to 500,000 Da (e.g., from 10,000 and 250,000 Da, between 20,000 and 125,000 Da, or between 40,000 and 60,000 Da).

In some examples, the copolymer blend includes PEDOT:PSS and P(PEGMA-b-PEG)-SH. In some examples, the copolymer blend includes a ratio of P(PEGMA-b-PEG)-SH to PEDOT:PSS is from 1:10 to 10:1 by weight (e.g., from 1:9, from 1:8, from 1:7, from 1:6, from 1:5, from 1:4, from 1:3, from 1:2, from 1:1, from 2:1, from 3:1, from 4:1, from 5:1, from 6:1, from 7:1, from 8:1, or from 9:1).

Also described herein are elastomeric materials including any one of the copolymer blends described herein. Provided also are coatings including one or more of the elastomeric materials described herein. Provided also is an object including any one of the elastomeric materials described herein, which can produced by molding, additive manufacturing, or 3D printing. Provided also are wearable devices including one of the elastomeric materials, films, objects, or coatings as described herein. In some examples, the wearable device is a wristband. In some examples, the wearable device is a monolithic conductive band. In some examples, the wearable device collects biopotential signals and/or electromyography signals.

### 2. Films Prepared from Intrinsically Conductive Elastomer Copolymer Blends

Described also herein is a film made from any one of the copolymer blends as described herein. In some examples, the film has a Young's Modulus from about 0.5 MPa to about 5.0 MPa (e.g., 1.0 MPa, 1.5 MPa, 2.0 MPa, 2.5 MPa, 3.0 MPa, 3.5 MPa, 4.0 MPa, 4.5 MPa, 5.0 MPa, 5.5 MPa, 6.0 MPa, 6.5 MPa, 7.0 MPa, 7.5 MPa, 8.0 MPa, 8.5 MPa, 9.0 MPa, or 9.5 MPa).

In some examples, the film has a thickness from about 0.1 mm to about 10 mm. In some examples, the film is a thin film having a thickness of up to about 3 mm, (e.g., 0.1 mm, 0.5 mm, 1.0 mm, 1.5 mm, 2.0 mm, or 2.5 mm). In some examples, the film described herein has an electrochemical impedance from about 0.2 Ohm to about 2 MOhm. In some examples, the film described herein has an electrical conductivity from about of 0.1 S cm⁻¹ to 1000 S cm⁻¹ S cm⁻¹. In some examples, the film exhibits 2% to 50% strain at break. In some examples, the film exhibits up to 20% strain at break.

### 3. Methods of Making Copolymer Blend

Provided also herein is a method of making any one of the copolymer blends described herein, including combining one or more of any one of the conductive polymers described herein with one or more of any one of the block copolymer counterions described herein, in a weight ratio of the conductive polymer to block copolymer counterion is 1:10 to 10:1 (e.g., 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, or 10:1). In some examples, the weight ratio of the conductive polymer to block copolymer counterion is 1:5 to 5:1 (e.g., 1:5, 1:2.5, 1:1, 2.5:1, or 5:1). In some examples, a bottlebrush block copolymer is optionally added to the copolymer blend. In some examples, the bottlebrush block copolymer is present in a weight ratio of bottlebrush block copolymer to conductive polymer and block copolymer counterion from 1:10 to 10:1. In some examples, the bottlebrush block copolymer is present in a weight ratio of bottlebrush block copolymer to conductive polymer and block copolymer counterion from 1:5 to 5:1

In some examples, the copolymer blend includes a weight ratio of P(PEGMA-b-PEG)-SH to PEDOT:PSS is 1:10 to 10:1 (e.g., 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, or 10:1). In some examples, the weight ratio of P(PEGMA-b-PEG)-SH to PEDOT:PSS is 1:5 to 5:1 (e.g., 1:5, 1:2.5, 1:1, 2.5:1, or 5:1).

### 4. Methods of Making Films Prepared from Intrinsically Conductive Elastomers

Provided also herein is a method of making any one of the films described herein, the method including combining one or more of any one of the copolymer blends described herein, optionally with any one of the crosslinkers described herein and any one of the photointiators describe herein to form a first solution, casting the first solution into a mold, and curing the first solution to form a film. Optionally, the curing step is a UV curing step.

Throughout this application, various publications are referenced. The disclosures of these publications in their entireties are hereby incorporated by reference into this application.

The examples below are intended to further illustrate certain aspects of the methods and compositions described herein, and are not intended to limit the scope of the claims.

### EXAMPLES

### Example 1: Intrinsically Conductive Elastomeric Materials Including Conductive Polymers and Block Copolymer Counterions

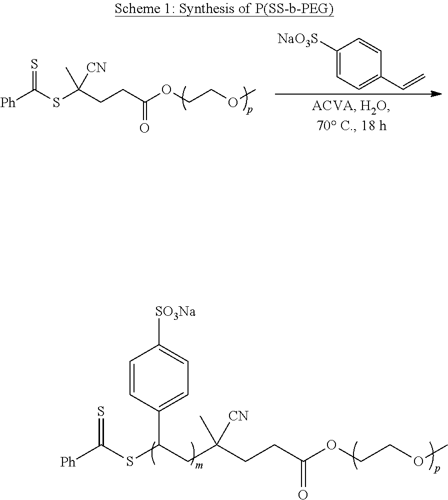

Scheme 1 provides a schematic of an example synthesis of P(SS-b-PEG). To a flask was added poly(ethylene glycol) 4-cyano-4-(phenylcarbonothioylthio)pentanoate (0.5 g, 0.05 mmol) (M_{w}˜10,000 Da), sodium 4-vinylbenzenesulfonate (1.55 g, 7.5 mmol), and 4,4′-azobis(4-cyanopentanoic acid) (3-10 mg, 0.01 mmol), in 10 mL water, and the reaction mixture was stirred at 70° C. for 18 hours. The resulting product was precipitated out of acetone and dried under vacuum. No leftover monomer was observed via ¹H NMR spectroscopy.

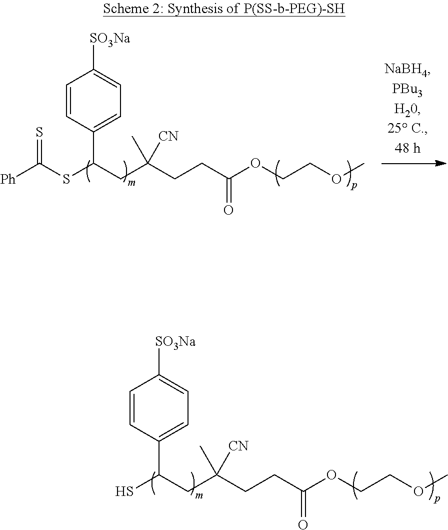

Scheme 2 provides a schematic of an example synthesis of P(SS-b-PEG)-SH. To a flask was added P(SS-b-PEG) (2 g, 0.065 mmol) (M_{w}˜30,000 Da), NaBH₄ (0.189 g, 5 mmol), tri n-butylphosphine (PBu₃) (1 mL, 4 mmol) in 10 mL water, and the reaction mixture was allowed to stir at 25° C. for 48 hours. The resulting product was precipitated out of acetone and dried under vacuum. No leftover monomer was observed by ¹H NMR spectroscopy.

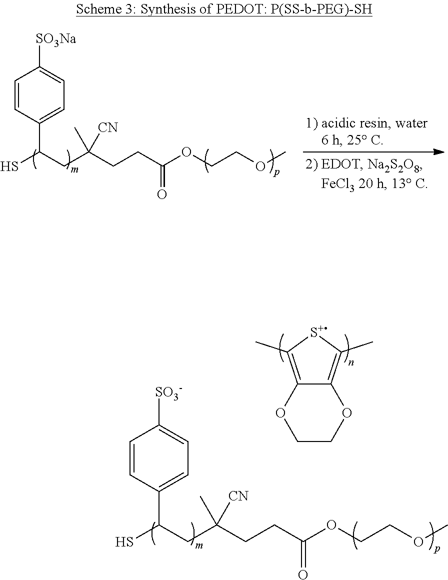

Scheme 3 provides a schematic of an example synthesis of PEDOT:P(SS-b-PEG)-SH. The P(SS-b-PEG)SH (0.14 g) (M_{w}˜30,000 Da) was pre-treated with an acid resin (3.6 mL) (AmberChrom™ 50WX4 200-400 Mesh (H+) Cation Exchange Resin, commercially available from Sigma-Aldrich, Inc. (St. Louis, MO)) in water, and allowed to stir at 25° C. for six hours. The acid-treated P(SS-b-PEG)SH was then removed from solution by filtration and dried under vacuum. Following the acid treatment, the acid-treated P(SS-b-PEG)SH (0.14 g, 0.82 wt %), 3,4-ethylenedioxythiophene (EDOT) (42 μL, 1.07 mmol), Na₂S₂O₈ (130 mg, 1.2 mmol), and 10 wt % FeCl₃ (30 μL, 0.2 mmol) in 15 mL water were allowed to stir at 13° C. for 20 hours. The resulting product was stirred in aqueous solution and filtered through a filter.

### Preparation of Films Including PEDOT:P(SS-b-PEG)-SH

To a flask was added 7 mL of 1.3 wt % PEDOT:P(SS-b-PEG)-SH aqueous solution, 2-hydroxy-4′-(2-hydroxyethoxy)-2-methylpropiophenone (0.009 g, 0.04 mmol) was added as a radical photoinitiator with PEG tetravinyl ether (0.053 g, 0.09 mmol) crosslinker, and the mixture was stirred for 30 minutes at 25° C. The solution was casted into a glass mold (6 mm×30 mm, 2 mm depth), purged with nitrogen for 240 seconds, and exposed to UV light for 120 minutes to cure the resulting material. Then, the solvent was slowly evaporated at room temperature to form free-standing films.

### Methods and Results for Characterization of Materials Properties

### 1. Tensile Test

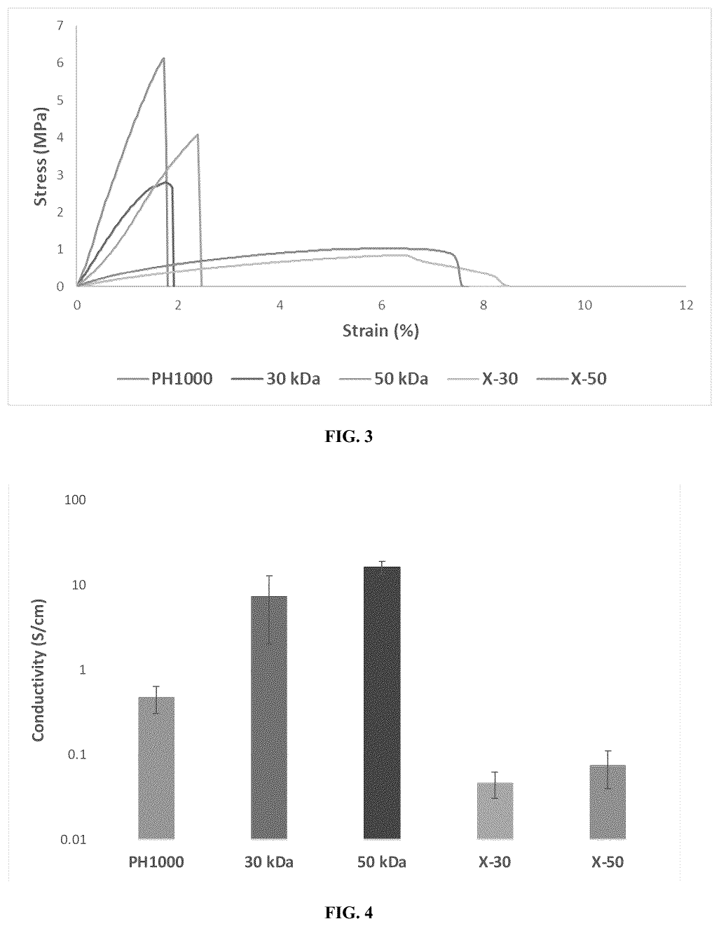

The tensile properties were measured on a dynamic mechanical analysis (DMA) tensile-compressive tester (DMA 850—TA Instruments) with a 0.1 g load cell. The two ends of the free-standing films were clamped using metal plates, and the upper clamp was used to stretch the samples at a strain rate of 2% min⁻¹ until the sample failed. The tensile machine was used to record stress and strain, as shown in FIG. 3. The elastic modulus was calculated from the slope of the stress-strain curve in the linear stress-strain region (FIG. 3).

FIG. 3 provides a plot showing the stress-strain curves of the different molecular weight of the copolymers and copolymers crosslinked with PEG tetravinyl ether as free-standing films (30 kDa corresponds to 30 kDa PEDOT:P(SS-b-PEG)-SH; 50 kDa corresponds to 50 kDa PEDOT:P(SS-b-PEG)-SH; X-30 corresponds to crosslinked 30 kDa PEDOT:P(SS-b-PEG)-SH, and X-50 corresponds to crosslinked 50 kDa PEDOT:P(SS-b-PEG)-SH). The free standing films prepared from copolymers and crosslinked copolymers described herein were also compared to free standing films prepared from PH100 (PEDOT:PSS, poly(2,3-dihydrothieno-1,4-dioxin)-poly(styrenesulfonate), also known as poly(3,4-ethylenedioxythiophene)-poly(styrenesulfonate), commercially available from Sigma-Aldrich, Inc. (St. Louis, MO)).

### 2. Electrochemical Impedance Spectroscopy (EIS)

To prepare samples for EIS, carbon conductive double-sided adhesive tape and free-standing film samples were punched by a 6 mm diameter puncher. The double-sided adhesive tape was then attached on the gold-plated brass electrode on a 3D printed electrode fixture. Then, the punched film sample was placed on the adhesive tape for at least 1 hour to create a firm conductive interface between film sample, adhesive tape, and the gold metal electrode. The electrode fixture was loaded to the 3D printed electrode holder of the glass beaker, which was filled with 1×phosphate-buffred saline (PBS) solution. Only the surface of the samples contacted the ionic solution. The counter electrode was a Platinum electrode (Pt) (3.0 mm diameter). The reference electrode was Platinum wire electrode. EIS measurements were performed on a PalmSens 4 potentiostat controlled and analyzed by PSTrace software, commercially available from PalmSens B.V. (Houten, Netherlands). For the electrochemical impedance spectroscopy (EIS), the scanning was from 0.1 to 1×10⁴ Hz at 0 V bias (vs. counter electrode) with 10 mV amplitude.

Table 1 provides fitted values for physical properties measured from free-standing films made from PH1000, 30 kDa, 50 kDa, X-30, and X-50 as described above.

TABLE 1

| | R₁ | Std | R₂ | Std | Q | Std | |
|---|---|---|---|---|---|---|---|
| | (Ω) | dev | (Ω) | dev | (μT) | dev | χ² |
| PH1000 | 48.20 | 9.57 | 1583.73 | 619.33 | 27.36 | 11.39 | 0.0029 |
| 30 kDa | 59.44 | 16.43 | 708.83 | 105.07 | 4.92 | 0.91 | 0.0003 |
| 50 kDa | 43.66 | 2.52 | 94.39 | 35.97 | 8.02 | 1.45 | 0.00007 |
| X-30 | 51.24 | 3.65 | 466.53 | 143.39 | 8.51 | 3.21 | 0.0015 |
| X-50 | 63.39 | 2.07 | 167.68 | 59.02 | 5.37 | 2.95 | 0.0055 |

The values of R₁ and R₂ are, respectively, the resistive contribution from 1×PBS electrolyte, and the interfacial resistance of free-standing films. Q is the constant phase element describing the nonideal capacitance of samples. χ² corresponds to the chi-squared distribution for the fit of the data for each film appearing in FIG. 3.

### 3. Electronic Conductivity

To prepare samples for conductivity measurements, glass slides were cut into 2.54 cm squares. The slides were then successively washed in soap, deionized water, acetone, and isopropyl alcohol (IPA) in an ultrasonic bath for 10 minutes each and dried with compressed air. The glass slides were then plasma treated at ˜30 W for 60 seconds(s) at a pressure of 200 mTorr under ambient air to remove any residual organic material and activate the surface. The PEDOT:P(SS-b-PEG)-SH solutions were spin-coated onto glass slides at a spin speed of 500 rpm (250 rpm s−1 ramp) for 120 seconds followed by 1000 rpm (500 rpm s−1 ramp) for 30 seconds. After spin-coating the samples were annealed on a hotplate at 120° C. for 15 minutes under a nitrogen flow. The resistance was measured by using the Filmetrics® 4-point probe. The thickness of the films was measured using a Bruker DektakXT profilometer to convert the resistance to conductivity. The conductivity σ was calculated inverse value of sheet resistance times sheet thickness and an average conductivity of three independently synthesized PEDOT:P(SS-b-PEG)-SH samples.

FIG. 4 provides a graph showing the effect of the molecular weight and crosslinking on the electrical conductivity of samples as measured by the Filmetrics® four-point probe.

### 4. Skin Contact Impedance

Material formulations passed the biocompatibility assessment for the electrode-skin impedance measurements (ESIMs). Preparation of samples for ESIM, Z-axis conductive double-sided adhesive tape and free-standing film samples were punched by a 6.5 mm diameter puncher. The double-sided adhesive tape was attached onto a gold-plated brass electrode. Then, a punched film sample was placed on the adhesive tape for at least 1 hour to create a firm conductive interface between sample, adhesive tape, and the gold metal electrode. The impedance data was collected by applying a potentiostat on the skin with 5 KPa applied on top of the electrodes.

### 5. Rheology of Ink

The viscosity of samples was measured using a parallel-plate geometry at 1% strain on an ARES Rheometer (TA Instruments, Wood Dale, IL, US) through a time sweep.

### 6. Water Stability Test

Different free-standing films were submerged in PBS solution at room temperature. The mass of the film was monitored over time.

### Example 2: Preparation of Intrinsically Conductive Elastomeric Materials Including Conductive Polymers, Block Copolymer Counterions, and Bottlebrush Block Copolymers

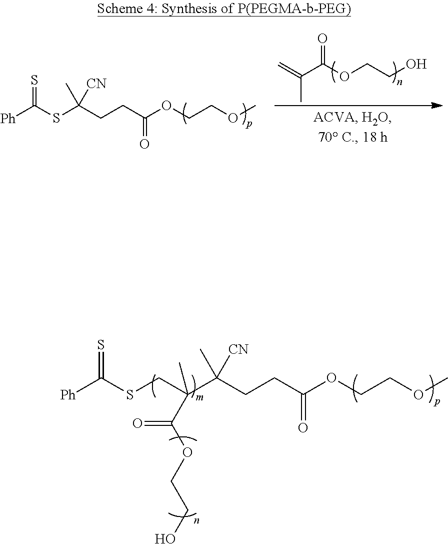

Scheme 4 provides a schematic of an example of the synthesis of P(PEGMA-b-PEG). To a flask was added poly(ethylene glycol) 4-cyano-4-(phenylcarbonothioylthio)pentanoate) (M_{w}˜10,000 Da) (0.5 g, 0.05 mmol), poly(ethylene glycol)methacrylate (1.26 g, 3.5 mmol), 4,4′-azobis(4-cyanopentanoic acid) (9 mg, 0.03 mmol), in 10 mL water, and the reaction mixture was stirred at 70° C. for 18 hours. The resulting product was subjected to dialysis in deionized water, and the solvent was removed in vacuo. The resulting product was then further dried under vacuum. No leftover monomer was observed via ¹H NMR spectroscopy.

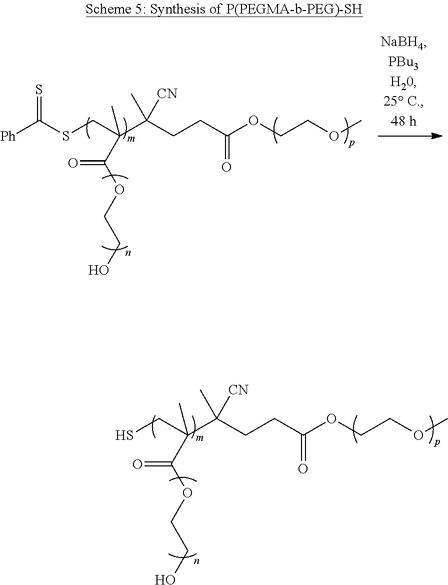

Scheme 5 provides a schematic of an example of the synthesis of P(PEGMA-b-PEG)-SH. To a flask was added P(PEGMA-b-PEG) (M_{w}˜30,000 Da) (2 g, 0.065 mmol), NaBH₄ (0.189 g, 5 mmol), PBu₃ (1 mL, 4 mmol), in 10 mL water, and the reaction mixture was allowed to stir at 25° C. for 48 hours. The resulting product was subject to dialysis in deionized water, and the solvent was removed in vacuo. The resulting product was then further dried under vacuum. No leftover monomer was observed by ¹H NMR spectroscopy.

### Preparation of a Blended Solution of PEDOT:PSS and P(PEGMA-b-PEG)-SH

Approximately 13 mg of PEDOT:PSS per 1 mL of solution (commercially available under the commercial name of Clevios™ PH 1000, from Heraeus Epurio LLC (Vandalia, Ohio)) was added to four different flasks. Approximately 13 mg, 32.5 mg, and 65 mg of P(PEGMA-b-PEG)-SH (M_{w}˜30,000 Da) were added to each of the four flasks, respectively, to yield a blended solution of P(PEGMA-b-PEG)-SH:PEDOT with weight ratios of P(PEGMA-b-PEG)-SH to PEDOT:PSS of about 1:1, 2.5:1, and 5:1, respectively.

### Preparation of Films Including a Blended Solution of PEDOT:PSS and P(PEGMA-b-PEG)-SH

To a flask was added 7 mL of 1.3 wt. % the blended aqueous solution of PEDOT:PSS and P(PEGMA-b-PEG)-SH, 2-hydroxy-4′-(2-hydroxyethoxy)-2-methylpropiophenone (0.009 g, 0.04 mmol) was added as a radical photoinitiator with PEG tetravinyl ether (0.053 g, 0.09 mmol) crosslinker, and the mixture was stirred for 30 minutes at 25° C. The solution was cast into a glass mold (6 mm×30 mm, 2 mm depth), purged with nitrogen for 240 seconds, and exposed to UV light for 120 minutes to cure the resulting material. Then, the solvent was slowly evaporated at room temperature in a fumehood to form free-standing films.

### Methods and Results for Characterization of Materials Properties

### 1. Tensile Test

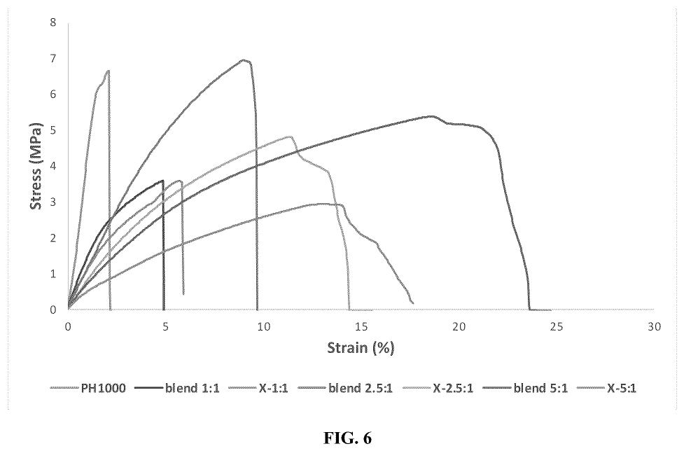

The tensile properties of the prepared films described above, that included a blended solution of PEDOT:PSS and P(PEGMA-b-PEG)-SH, were measured on a dynamic mechanical analysis (DMA) tensile-compressive tester (DMA 850—TA Instruments) with a 0.1 g load cell. The two ends of the free-standing films were clamped using metal plates, and the upper clamp was used to stretch the samples at a strain rate of 2% minute⁻¹ until the sample failed. The tensile-compressive tester was used to record stress and strain. The elastic modulus was calculated from the slope of the stress-strain curve, shown in FIG. 6, in the linear stress-strain region.

FIG. 6 provides a plot showing the stress-strain curves of the different weight ratio blends of the copolymers and weight ratio blends copolymers crosslinked with PEG tetravinyl ether as free-standing films (blend 1:1 corresponds to the film prepared from the 1:1 weight ratio of P(PEGMA-b-PEG)-SH to PEDOT:PSS; X-1:1 corresponds to the film prepared from the 1:1 weight ratio of P(PEGMA-b-PEG)-SH to PEDOT:PSS crosslinked with PEG tetravinyl ether; blend 2.5:1 corresponds to the film prepared from the 2.5:1 weight ratio of P(PEGMA-b-PEG)-SH to PEDOT:PSS; X-2.5:1 corresponds to the film prepared from the 2.5:1 weight ratio of P(PEGMA-b-PEG)-SH to PEDOT:PSS crosslinked with PEG tetravinyl ether; blend 5:1 corresponds to the film prepared from the 5:1 weight ratio of P(PEGMA-b-PEG)-SH to PEDOT:PSS; and X-5:1 corresponds to the film prepared from the 5:1 weight ratio of P(PEGMA-b-PEG)-SH to PEDOT:PSS crosslinked with PEG tetravinyl ether). The free standing films prepared from copolymers and crosslinked copolymers described herein were also compared to free standing films prepared from PH100 (PEDOT:PSS, poly(2,3-dihydrothieno-1,4-dioxin)-poly(styrenesulfonate), also known as poly(3,4-ethylenedioxythiophene)poly(styrenesulfonate), commercially available from Sigma-Aldrich, Inc. (St. Louis, MO)).

Table 2 below shows the mechanical properties of the films prepared from the polymer blends described above.

TABLE 2

| | | | Young's | | | |
|---|---|---|---|---|---|---|
| | Strain | Std | modulus | Std | Toughness | Std |
| | (%) | dev | (MPa) | dev | (kPa) | dev |
| PH1000 | 2.16 | 0.35 | 4.48 | 0.37 | 96.62 | 37.75 |
| blend 1:1 | 4.72 | 0.83 | 1.78 | 0.57 | 106.21 | 7.81 |
| X-1:1 | 5.15 | 1.02 | 1.07 | 0.11 | 107.89 | 32.83 |
| blend 2.5:1 | 9.59 | 0.86 | 1.10 | 0.07 | 426.17 | 62.85 |
| X-2.5:1 | 11.85 | 0.13 | 0.72 | 0.13 | 278.40 | 88.56 |
| blend 5:1 | 20.24 | 1.32 | 0.78 | 0.17 | 818.95 | 183.51 |
| X-5:1 | 14.92 | 0.68 | 0.48 | 0.13 | 282.29 | 25.15 |

### 2. Electrochemical Impedance Spectroscopy (EIS)

To prepare samples for EIS, carbon conductive double-sided adhesive tape and free-standing films samples were punched by a 6 mm diameter puncher. The adhesive tape was attached on a gold-plated brass electrode on a 3D printed electrode fixture. Then, the punched film sample was placed on the adhesive tape for at least 1 hour to create a firm conductive interface between sample, adhesive tape, and the gold metal electrode. The electrode fixture was loaded to the 3D printed electrode holder of the glass beaker, which was filled with 1×PBS solution. Only the surface of the samples were allowed to contact the 1×PBS solution. The counter electrode was a Platinum electrode (Pt) (3.0 mm diameter), and the reference electrode was a Platinum wire electrode. EIS measurements were performed on a PalmSens 4 potentiostat controlled and analyzed by the PSTrace software. Electrochemical impedance spectroscopy (EIS) was performed by scanning from 0.1 to 1×10⁴ Hz at 0 V bias (vs. counter electrode) with 10 mV amplitude.

Table 3 provides fitted values for physical properties measured from free-standing films made from PH1000, blend 1:1, X-1:1, blend 2.5:1, X-2.5:1, blend 5:1, X-5:1 as described herein.

TABLE 3

| | R₁ | Std | R₂ | Std | Q | Std | |
|---|---|---|---|---|---|---|---|
| | (Ω) | dev | (Ω) | dev | (uT) | dev | χ² |
| PH1000 | 48.20 | 14.46 | 1583.73 | 619.33 | 16.19 | 11.39 | 0.0027 |
| blend 1:1 | 72.00 | 13.90 | 853.50 | 193.05 | 4.77 | 1.76 | 0.0021 |
| X-1:1 | 63.00 | 2.54 | 585.63 | 164.96 | 2.62 | 0.57 | 0.0015 |
| blend 2.5:1 | 41.88 | 3.31 | 374.57 | 117.23 | 4.67 | 2.50 | 0.0037 |
| X-2.5:1 | 40.16 | 0.72 | 230.37 | 122.39 | 16.99 | 5.21 | 0.0013 |
| blend 5:1 | 44.27 | 7.74 | 447.05 | 136.95 | 15.57 | 7.71 | 0.0092 |
| X-5:1 | 56.11 | 2.54 | 291.30 | 165.49 | 23.18 | 6.27 | 0.0009 |

The values of R₁ and R₂ are, respectively, the resistive contribution from 1×PBS electrolyte, and the interfacial resistance of free-standing films. Q is the constant phase element describing the nonideal capacitance of samples. χ² corresponds to the chi-squared distribution for the fit of the data for each film appearing in FIG. 6.

### 3. Electronic Conductivity

To prepare samples for conductivity measurements, glass slides were cut into 2.54 cm squares. The slides were then successively washed in soap, deionized water, acetone, and isopropyl alcohol (IPA) in an ultrasonic bath for 10 minutes each and dried with compressed air. The glass slides were then plasma treated at ˜30 W for 60 seconds(s) at a pressure of 200 mTorr under ambient air to remove any residual organic material and activate the surface. The solutions of PEDOT:PSS and P(PEGMA-b-PEG)-SH were spin-coated onto glass slides at a spin speed of 500 rpm (250 rpm s⁻¹ ramp) for 120 seconds followed by 1000 rpm (500 rpm ⁻¹ ramp) for 30seconds. After spin-coating, the samples were annealed on a hotplate at 120° C. for 15 minutes under a nitrogen flow. The resistance was measured by using the Filmetrics® 4-point probe. The thickness of the films was measured using a Bruker DektakXT profilometer to convert the resistance to conductivity. The conductivity σ was calculated inverse value of sheet resistance times sheet thickness and an average conductivity of three independently synthesized samples.

### 4. Skin Contact Impedance

Material formulations were passed the biocompatibility assessment for the electrode-skin impedance measurements (ESIMs). To prepare samples for ESIM, Z-axis conductive double-sided adhesive tape and free-standing film samples were punched by a 6.5 mm diameter puncher. The adhesive tape was attached on the gold-plated brass electrode. Then, the punched film sample was placed on the adhesive tape for at least 1 hour to create a firm conductive interface between sample, adhesive tape, and the gold metal electrode. The impedance data was collected by applying a potentiostat on the skin with 5 KPa applied on top of the electrodes.

### 5. Rheology of Ink

The viscosity of samples was measured using a parallel-plate geometry at 1% strain on an ARES Rheometer (TA Instruments, Wood Dale, IL, US) through a time sweep.

### 6. Water Stability Test

Different free-standing films were submerged in PBS solution at room temperature. The mass of the film was monitored over time.

The compositions and methods of the appended claims are not limited in scope by the specific compositions and methods described herein, which are intended as illustrations of a few aspects of the claims and any compositions and methods that are functionally equivalent are within the scope of this disclosure. Various modifications of the compositions and methods in addition to those shown and described herein are intended to fall within the scope of the appended claims. Further, while only certain representative compositions, methods, and aspects of these compositions and methods are specifically described, other compositions and methods are intended to fall within the scope of the appended claims. Thus, a combination of steps, elements, components, or constituents can be explicitly mentioned herein; however, all other combinations of steps, elements, components, and constituents are included, even though not explicitly stated.

What is claimed is:

1. A composition comprising:
a conductive polymer; and
a polymer counterion;
wherein a weight ratio of the conductive polymer and the polymer counterion is from 1:10 to 10:1.

2. The composition of claim 1, wherein the polymer counterion is a block copolymer counterion.

3. The composition of claim 1, wherein the conductive polymer has one or more side chains comprising functional groups selected from the group consisting of thiols, carboxylic acids, amines, hydroxyls, ionic groups, or a combination thereof.

4. The composition of claim 1, wherein the conductive polymer comprises poly(3,4-ethylenedioxythiophene) (PEDOT), polypyrrole (PPy), polyaniline (PANI), or a combination thereof.

5. The composition of claim 1, wherein the composition further comprises a bottlebrush block copolymer.

6. The composition of claim 5, wherein the bottlebrush block copolymer comprises PEGMA-b-PEG.

7. The composition of claim 1, wherein the polymer counterion comprises one or more of poly(styrene sulfonate) (PSS), poly(ethylene glycol) 4-cyano-4-(phenylcarbonothioylthio)pentanoate, poly(ethylene oxide)monomethacrylate (PEGMA), (P(SS-b-PEG)), or a combination thereof.

8. The composition of claim 1, wherein the polymer counterion comprises at least one crosslinkable chain end.

9. The composition of claim 8, wherein the crosslinkable chain end is a thiol.

10. The composition of claim 1, further comprising one or more PEG vinyl ether crosslinkers and one or more photoinitiators.

11. The composition of claim 1, wherein the conductive polymer comprises PEDOT and the polymer counterion comprises P(SS-b-PEG)-SH.

12. A copolymer blend comprising:
a conductive polymer comprising one or more of poly(3,4-ethylenedioxythiophene) (PEDOT), polypyrrole (PPy), polyaniline (PANI), or a combination thereof; and
a polymer counterion comprising one or more of poly(styrene sulfonate) (PSS), poly(ethylene glycol) 4-cyano-4-(phenylcarbonothioylthio)pentanoate, poly(ethylene oxide)monomethacrylate (PEGMA), (P(SS-b-PEG)), or a combination thereof.

13. The copolymer blend of claim 12, further comprising a bottlebrush block copolymer.

14. The copolymer blend of claim 13, wherein the bottlebrush block copolymer comprises PEGMA-b-PEG.

15. The copolymer blend of claim 13, wherein at least one of the polymer counterion or the bottlebrush block copolymer comprises a thiol chain end.

16. The copolymer blend of claim 15, wherein the thiol chain end is crosslinked with a PEG vinyl ether.

17. A copolymer blend comprising PEDOT:PSS and P(PEGMA-b-PEG)-SH.

18. The copolymer blend of claim 17, wherein a ratio of P(PEGMA-b-PEG)-SH to PEDOT:PSS is from 1:10 to 10:1 by weight.

19. An elastomeric material comprising the copolymer blend of claim 17.

20. A film comprising the copolymer blend of claim 17.

21. An object comprising the elastomeric material of claim 19, wherein the object is produced by molding, additive manufacturing, or 3D printing.

22. A wearable device comprising the film of claim 20.

23. The wearable device of any claim 22, wherein the wearable device collects at least one of biopotential signals or electromyography signals.
